(19) United States
(12) Patent Application Publication
(10) Pub. No.: US 2011/0143340 A1
(43) Pub. Date: Jun. 16, 2011

(54) NON-INVASIVE ISOLATION OF FETAL NUCLEIC ACID

(75) Inventors: Wen-Hua Fan, San Diego, CA (US); Irene Yasuda, San Diego, CA (US); Karena Kosco, Carlsbad, CA (US); Ram Bhatt, San Diego, CA (US)

(73) Assignee: BIOCEPT, INC., San Diego, CA (US)

(21) Appl. No.: 12/740,418

(22) PCT Filed: Oct. 30, 2008
(86) PCT No.: PCT/US2008/081780

§ 371 Date: Mar. 3, 2011

Related U.S. Application Data
(60) Provisional application No. 60/984,698, filed on Nov. 1, 2007.

(51) Int. Cl. C12Q 1/68; C12N 5/073

(52) U.S. Cl. 435/6.1; 435/325

(57) ABSTRACT

The present invention provides compositions comprising a solution of a compound useful for lysing biological cells and methods for using the compositions to lyse biological cells and to isolate nucleic acid.

### CROSS-REFERENCE TO RELATED APPLICATIONS

This application claims the benefit of priority under 35USC §119(e) to U.S. Provisional Patent Application Ser. No. 60/984,698 filed Nov. 1, 2007, the disclosure of which is incorporated herein by reference in its entirety.

### BACKGROUND OF THE INVENTION

Prenatal testing or screening is usually performed to determine the gender of the fetus or to detect genetic disorders and/or chromosomal abnormalities in the fetus during pregnancy. As of today, over 4000 genetic disorders, caused by one or more faulty genes, have been recognized. Some examples include Cystic Fibrosis, Huntington's Disease, Beta Thalassaemia, Myotonic Dystrophy, Sickle Cell Anemia, Porphyria, and Fragile-X-Syndrome. Chromosomal abnormality is caused by aberrations in chromosome numbers, duplication or absence of chromosomal material, and by defects in chromosome structure. Examples of chromosomal abnormalities are trisomies, e.g., trisomy 16, a major cause of miscarriage in the first trimester, trisomy 21 (Down syndrome), trisomy 13 (Patau syndrome), trisomy 18 (Edwards syndrome), Klinefelter's syndrome (47, XXY), (47, XYY), and (47, XXX); the absence of chromosomes (monosomy), e.g., Turner syndrome (45, X0); chromosomal translocations, deletions and/or microdeletions, e.g., Robertsonian translocation, Angelman syndrome, DiGeorge syndrome and Wolf-Hirschhorn Syndrome.

Currently available prenatal genetic tests usually involve invasive procedures. For example, chorionic villus sampling (CVS) performed on a pregnant woman around 10-12 weeks into the pregnancy and amniocentesis performed at around 14-16 weeks all contain invasive procedures to obtain the sample for testing chromosomal abnormalities in a fetus. Fetal cells obtained via these sampling procedures are usually tested for chromosomal abnormalities using cytogenetic or fluorescent in situ hybridization (FISH) analyses.

While these procedures can be useful for detecting chromosomal aberrations, they have been shown to be associated with the risk of miscarriage. Therefore amniocentesis or CVS is only offered to women perceived to be at increased risk, including those of advanced maternal age (>35 years), those with abnormal maternal serum screening or those who have had a previous fetal chromosomal abnormality. As a result of these tests the percentage of women over the age of 35 who give birth to babies with chromosomal aberrations such as Down syndrome has drastically reduced. However, lack of appropriate or relatively safe prenatal testing or screening for the majority of pregnant women has resulted in about 80% of Down syndrome babies born to women under 35 years of age.

Thus there is a need for non-invasive screening tests for the general population of pregnant women, especially tests directed to identifying fetal chromosomal aberrations as well as other genetic variations, disorders or diseases. This requires non-invasive techniques of isolating fetal nucleic acid that can be used for prenatal genetic screening.

### SUMMARY OF THE INVENTION

The present invention is based, in part, on the discovery of a compound useful for lysing biological cells. Accordingly, the present invention provides compositions comprising a solution of the compound and methods for using the compositions to lyse biological cells and to isolate nucleic acid.

In one embodiment of the invention, it provides a composition comprising a solution of S-[(2-Guanidino-4-thiazoyl)methyl]-isothiourea (GTMI), or a salt thereof. The concentration of GTMI in the solution range from about 0.1 mM to about 500 mM and the pH of the solution ranges from about pH 6 to about pH 9.

In another embodiment of the invention, it provides a method of lysing cells in a biological sample. The method comprises contacting the biological sample containing one or more cells with a composition of the invention.

In yet another embodiment of the invention, it provides a method of preferentially lysing apoptotic cells in a biological sample. The method comprises contacting the biological sample containing apoptotic and non-apoptotic cells with a lysing agent for a period of time such that the apoptotic cells are preferentially lysed over the non-apoptotic cells.

In still another embodiment of the invention, it provides a method of preferentially lysing fetal cells in a maternal biological sample. The method comprises contacting the maternal biological sample containing fetal cells with a lysing agent for a period of time such that the fetal cells are preferentially lysed over maternal cells in the biological sample.

In yet another embodiment of the invention, it provides a method of isolating nucleic acids from fetal cells. The method comprises contacting a maternal biological sample containing fetal cells with a lysing agent for a period of time, such that the fetal cells are preferentially lysed over maternal cells in the biological sample, to form a lysing mixture. The nucleic acid is isolated from the lysing mixture.

In yet another embodiment of the invention, it provides a method of identifying the genetic composition of a subject. The method comprises lysing cells in a biological sample of a subject according to a method of the invention, to form a lysing mixture, and identifying the genetic composition of the subject based on a nucleic acid contained in the lysing mixture.

In yet another embodiment of the invention, it provides a method of identifying the genetic composition of a fetus. The method comprises lysing fetal cells in a maternal biological sample according to a method of the invention, to form a lysing mixture, and identifying the genetic composition of the fetus based on a nucleic acid contained in the lysing mixture.

### BRIEF DESCRIPTION OF THE FIGURES

FIG. 1 shows the chemical structure of S-[(2-Guanidino-4-thiazoyl)methyl]-isothiourea hydrochloride (GTMI).

FIG. 2 shows a flow chart depicting exemplary steps of a method of the invention.

FIG. 3 depicts an exemplary gel used for size fractionation separation of fetal and maternal DNA.

FIG. 4 shows electropherogram results of PCR analysis of fetal DNA. FIG. 4A shows the results using chromosome 4 primers and FIG. 4B shows the results using chromosome 21 primers.

### DETAILED DESCRIPTION OF THE INVENTION

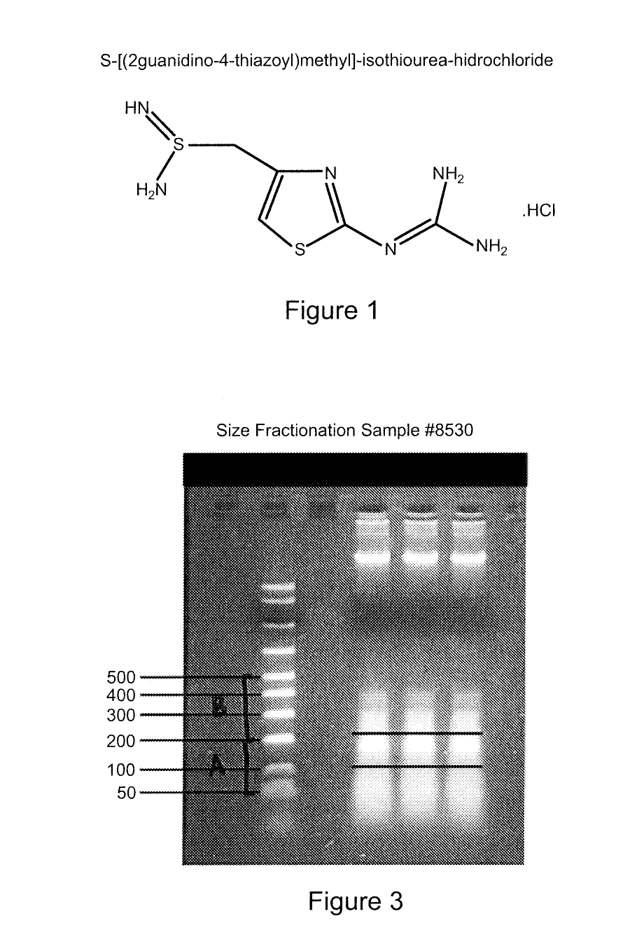

The present invention is based, in part, on the discovery of a compound useful for lysing biological cells. According to one aspect of the present invention, it provides a composition comprising a solution of S-[(2-Guanidino-4-thiazoyl)methyl]-isothiourea (GTMI), or a salt thereof. The chemical structure of GTMI hydrochloride is provided in FIG. 1. As used herein, the terms “S-[(2-Guanidino-4-thiazoyl)methyl]-isothiourea” and “GTMI” are used interchangeably and refer to both the compound as well as any salt thereof. Any suitable salt of the compound can be used in the compositions of the invention. Examples of salts that can be used include, but are not limited to, halides, for example, chloride, bromide, or iodide; acetate; sulfate; isocyanates; isothiocyanate; and phosphates.

The concentration of GTMI in the solution can range from about 0.1 mM to about 500 mM in an aqueous solvent, an organic solvent, or a combination thereof. In one embodiment, the concentration of GTMI in the solution is from about 0.1 mM to about 450 mM, 400 mM, 350 mM, 300 mM, 250 mM, 200 mM, 150 mM, 100 mM or 50 mM. In another embodiment, the concentration of GTMI in the solution ranges from about 0.5 mM to about 500 mM, 450 mM, 400 mM, 350 mM, 300 mM, 250 mM, 200 mM, 150 mM, 100 mM, or 50 mM. In yet another embodiment, the concentration of GTMI in the solution ranges from about 1 mM to about 500 mM, 450 mM, 400 mM, 350 mM, 300 mM, 250 mM, 200 mM, 150 mM, 100 mM, or 50 mM. In still another embodiment, the concentration of GTMI in the solution ranges from about 0.5 mM to about 50 mM, 45 mM, 40 mM, 35 mM, 30 mM, 25 mM, 20 mM, 15 mM, 10 mM, or 5 mM. In yet another embodiment, the concentration of GTMI in the solution ranges from about 1 mM to about 50 mM, 45 mM, 40 mM, 35 mM, 30 mM, 25 mM, 20 mM, 15 mM, 10 mM, or 5 mM. Examples of organic solvents include, but are not limited to, DMSO and DMF.

In general, the pH of the solution can range from about pH 6 to about pH 9. In one embodiment, the pH of the solution ranges from about pH 6 to about pH 8.5, pH 8, pH 7.5, or pH 7. In another embodiment, the pH of the solution ranges from about pH 6.5 to about pH 9, pH 8.5, pH 8, pH 7.5, or pH 7. In yet another embodiment, the pH of the solution ranges from about pH 7 to about pH 9, pH 8.5, pH 8, or pH 7.5.

In addition to the solution of GTMI, or a salt thereof, the composition may comprise other components including, but not limited to, a buffer, an additional lysing agent, a surfactant or detergent. Exemplary additional components as well as the concentrations at which they are present would be known to one of skill in the art and the following are only non-limiting examples of the additional components and the concentrations at which they are present.

In one embodiment, the composition further comprises a buffer. The buffer can be present at any suitable concentration required to maintain the pH of the solution at a desired pH in the range from about pH 6 to about pH 9. The concentration of the buffer can range from about 5 mM to about 100 mM, 150 mM, 200 mM, 250 mM, 300 mM, 350 mM, 400 mM, 450 mM, or 500 mM. Alternatively, the buffer can range from about 5 mM to about 20 mM, 30 mM, 40 mM, 50 mM, 60 mM, 70 mM, 80 mM 90 mM or 100 mM; or from about 10 mM to about 20 mM, 30 mM, 40 mM, 50 mM, 60 mM, 70 mM, 80 mM, 90 mM or 100 mM.

Examples of buffers that can be present in the compositions of the invention include, but are not limited to, ([tris(hydroxymethyl)methyl]amino)propanesulfonic acid (TAPS); N,N-bis(2-hydroxyethyl)glycine (Bicine); tris(hydroxymethyl)methylamine (Tris); N-tris(hydroxymethyl)methylglycine (Tricine); N-2-hydroxyethyl-1-piperazineethanesulfonic acid (HEPES); 2-([Tris(hydroxymethyl)methyl]amino)ethanesulfonic acid (TES); (N-morpholino)propanesulfonic acid (MOPS); piperazine-N,N′-bis(2-ethanesulfonic acid) (PIPES); dimethylarsinic acid (Cacodylate); 2-(N-morpholino)ethanesulfonic acid (MES); N-(2-hydroxyethyl)piperazine-N′-2-hydroxypropane-sulfuric acid (HEPPSO); N,N′-Bis(2-hydroxyethyl)-2-aminoethanesulfonic acid (BES); and Phosphate (e.g., Sodium phosphate).

In another embodiment, the composition further comprises an additional lysing agent, a salt, a surfactant or detergent. The salt can be any suitable salt, including but not limited to, sodium acetate, sodium chloride, sodium citrate, sodium formate and sodium phosphate. Exemplary concentrations of the salts include, but are not limited to, from about 5 mM to about 500 mM, 400 mM, 300 mM, 250 mM, 200 mM, 150 mM, 100 mM, or 50 mM; or from about 10 mM to about 300 mM, 250 mM, 200 mM, 150 mM, 100 mM, or 50 mM. The detergent can be any detergent known to one of skill in the art. In an exemplary embodiment, the detergent is a non-ionic detergent. Examples of non-ionic detergents include, but are not limited to, Triton X-100, Triton X-114, Triton X-405, Dodecyl-beta-D-glucopyrnaside, Dodecyl-beta-D-maltoside, n-Decyl-beta-D-maltopyranside, n-Dodecanoylsucrose, n-Heptyl-beta-D-thioglucopyranoside, n-Hexyl-beta-D-glucopyranside, n-Octanoylsucrose, IGEPAL, Pluronic F-68, HECAMEG, ELUGENT, PLURINIC F-127, Big CHAP, Saponin, Tween-20, Zwittergent 308, 312, 316, and n-Dodecyl-octaethylene glycol (C12E8).

In yet another embodiment, the composition further comprises Vitamin E. Vitamin E can be present in the composition at a concentration range from about 0.1 mM to about 0.3 mM, 0.4 mM, 0.5 mM, 0.6 mM, 0.7 mM, 0.8 mM, 0.9 mM, 1.0 mM, 1.2 mM, 1.5 mM, or 2 mM.

The compositions of the present invention can also comprise any combination of the components identified above. For example, the composition can comprise GTMI, or a salt thereof, and any combination of a buffer and/or detergent. In one embodiment, the composition of the invention comprises a salt of GTMI, e.g., a halide salt of GTMI, such as GTMI hydrochloride, and a buffer, e.g., HEPES. The pH of the solution can, for example, range from about pH 7.0 to about pH 8.0, and the concentration of HEPES in the solution can range from about 10 mM to about 50 mM.

In another embodiment, the composition of the invention comprises a salt of GTMI, e.g., a halide salt of GTMI, such as GTMI hydrochloride, a buffer, e.g., HEPES, a non-ionic detergent, for example, Triton X-100, and optionally, Vitamin E. The concentration of the various components of the composition can vary, and determining the appropriate concentrations is known to one of skill in the art. Exemplary concentrations of HEPES range from about 10 mM to about 200 mM, and that of Vitamin E range from about 0.2 mM to about 1 mM. Further, the composition can comprise about 0.1% to 1, 2, 3, 4, 5, 6, 7, 8, 9 or 10% Triton X-100. In yet another embodiment, the composition comprises about 0.5% to about 1, 1.5, 2, 2.5, 3, 3.5, 4, 4.5 or 5% Triton X-100.

In still another embodiment, the composition comprises any of the following solutions:

i. 2.0 mM solution of GTMI, or a salt thereof, in 200 mM HEPES buffer, pH 7.2;
ii. 2.0 mM solution of GTMI, or a salt thereof, in 200 mM HEPES buffer and 1% Triton X-100;
iii. 2.0 mM solution of GTMI, or a salt thereof, in 200 mM HEPES buffer and 0.4 mM Vitamin E; or
iv. 2.0 mM solution of GTMI, or a salt thereof, in 200 mM HEPES buffer, 0.4 mM Vitamin E and 1% Triton X-100. These solutions could, optionally, include a salt, e.g., NaCl.

According to another aspect of the present invention, it provides a method of lysing cells in a biological sample. The method comprises contacting a biological sample containing one or more cells with any composition of the invention. The biological sample can be any cell or tissue sample from a prokaryotic or eukaryotic organism. Exemplary biological samples that can be used in the methods of the invention include, but are not limited to, blood, plasma, serum, bone marrow, tear, aqueous humour, vitreous humour, saliva, spinal fluid, urine, sputum, mucus, pleural fluid, synovial fluid, sweat, semen, menses, amniotic fluid, cervical mucus or chorionic villus sample.

According to yet another aspect of the present invention, it provides a method of preferentially lysing apoptotic cells in a biological sample. The method comprises contacting a biological sample, e.g., containing apoptotic and non-apoptotic cells with a lysing agent for a period of time such that the apoptotic cells are preferentially lysed over the non-apoptotic cells. In general, apoptotic cells are cells that are susceptible to, immediately prior to, or in the process of cell death, e.g., programmed cell death or displaying any common characteristic of cell death or apoptosis.

According to still another aspect of the present invention, it provides a method of preferentially lysing fetal cells in a maternal biological sample. The method comprises contacting a maternal biological sample, e.g., containing fetal cells with a lysing agent for a period of time such that the fetal cells are preferentially lysed over maternal cells in the biological sample. Exemplary maternal biological samples include, but are not limited to, blood, plasma, serum, urine, cervical mucus, amniotic fluid, or chorionic villus sample.

Any suitable lysing agent can be used for preferentially lysing apoptotic cells or fetal cells or both. In one embodiment, a suitable lysing agent is a composition provided by the present invention. In another embodiment, a suitable lysing agent is any lysing agent with above average, e.g., substantially strong lysing activity. In yet another embodiment, a suitable lysing agent is any lysing agent combining the structure characteristics of at least two commonly used lysing agents. Examples of lysing agents include, but are not limited to, GTMI, or a salt thereof, guanidinium hydrochloride, guanidinium isothiocyanate; urea, lithium ferricyanide, sodium ferricyanide and thiocyanate, potassium ferricyanide and thiocyanate, ammonium chloride, diethylene glycol, Zap-Oglobin and commonly used detergents such as Tritons and NP-40, etc.

In one exemplary embodiment, GTMI, or a salt thereof, can be used at a concentration ranging from 0.1 mm to about 500 mM. In another exemplary embodiment, the concentration of GTMI, or a salt thereof, can range from about 0.5 mM to about 100 mM. In yet another exemplary embodiment, the concentration of GTMI, or a salt thereof, can range from about 1 mM to about 25 mM. In still another exemplary embodiment, the concentration of GTMI, or a salt thereof, can range from about 1 mM to about 5 mM.

Without being bound to any theory, it is believed that apoptotic and/or fetal cells are more sensitive to lysing agent, e.g., lysing agents provided by the present invention than non-apoptotic or maternal cells, therefore proper conditions can be set up to preferentially breakdown apoptotic and/or fetal cells in the presence of non-apoptotic and/or maternal cells. For example, according to the present invention apoptotic and/or fetal cells can be preferentially lysed at a concentration lower than the concentration required to lyse non-apoptotic and/or maternal cells or during a period of time shorter than the time required to lyse non-apoptotic and/or maternal cells (if the same concentration of lysing agent is used). According to the present invention, various factors associated with a lysis condition can be varied to preferentially lyse either apoptotic or fetal cells. Exemplary factors, including, but not limited to, time period of the lysis reaction, concentration of the lysing agent, nature of the lysing agent, pH of the lysing solution and temperature at which the lysis reaction is carried out can be varied so as to achieve preferential lysing of the apoptotic or fetal cells, but not that of the non-apoptotic or maternal cells.

The factors may be varied vis-à-vis one another to achieve the desired level of lysis. For example, the stronger the lysing agent, the lower the concentration needed as compared to a relatively weaker lysing agent. Alternatively, the stronger the lysing agent, the less would be the time period of the reaction to achieve the same level of lysis as with a weaker lysing agent. Other factors such as concentration and time, concentration and temperature of the reaction, or time and temperature of the reaction can also be varied to achieve the desired lysis level.

The desired level of lysis can vary depending on the ratios of, for example the apoptotic and non-apoptotic, or the fetal and maternal cells in the biological sample. In one embodiment, less than about 25%, or 20%, or 15%, or 10%, or 5% or 3% or 2% or 1% of non-apoptotic cells or maternal cells are lysed. In another embodiment, at least 0.1%, 1%, 5%, 10%, 15%, 20%, 30%, 40%, 50%, 60%, 70%, 80%, or 90% of apoptotic cells or fetal cells are lysed.

As described above, various factors can be varied to achieve the desired level of lysis. In one embodiment, the biological sample is contacted with about a 0.1 mm to about a 500 mM GTMI solution for about 1-10 seconds, at the high end of the concentration range to about an hour at the low end of the concentration range. In another embodiment, the biological sample is contacted with about a 1 mM to about 25 mM GTMI solution for about 5 minutes, at the higher end of the concentration range to about 30 minutes at the lower end of the concentration range. In yet another embodiment, the biological sample is contacted with about a 1 mM to about 5 mM GTMI solution for about 10-30 minutes. Such variations and manipulations are within the knowledge of one of skill in the art.

According to yet another aspect of the present invention, it provides a method of isolating nucleic acids from fetal cells. The method comprises contacting a maternal biological sample containing fetal cells with a lysing agent for a period of time, such that the fetal cells are preferentially lysed over maternal cells in the biological sample, to form a lysing mixture. The nucleic acid is isolated from the lysing mixture.

The nucleic acid can be isolated from the lysing mixture by any means known in the art. In one embodiment, the nucleic acid is isolated by any suitable means from a supernatant obtained by centrifuging the lysing mixture. The supernatant could, optionally, be further treated before isolating the nucleic acid. For example, the supernatant could be treated with a reagent, e.g., proteinase K that digests proteins and helps clean or purify the nucleic acid in the lysing mixture. Such a reagent, if used, is deactivated, e.g., by heating the sample to about 95° C. The nucleic acid can then be further purified by extractions with, for example chloroform and phenol, and precipitated in ethanol. The nucleic acid pellet can then be suspended in nuclease free water and used for further genetic analysis. Alternatively, the nucleic acid from the supernatant can be cleaned using a commercially available kit, e.g., Roche's Apoptotic DNA Ladder kit, or QIAMP DNA Blood Mini Kit, or Roche's MagNA Pure LC DNA Kit 1.

In another embodiment, the nucleic acid is isolated from the lysing mixture by contacting the lysing mixture with a ligand for nucleic acids. The ligand, can, for example, be coated or immobilized on a solid surface. The ligand can be coated or immobilized on the solid surface either directly, or indirectly, for example, via a linker. Methods for attaching ligands to solid surfaces are well known to those skilled in the art and any method now known, or later developed, can be used. In one embodiment, the solid surface is a population of magnetic particles, a particle contained in a column, e.g., a resin column, a surface of a microchannel, a microwell, a plate, a filter, a membrane, or a glass slide.

In yet another embodiment, the ligand can be coated on the surface of an apparatus, e.g., a microflow apparatus. An exemplary microflow apparatus comprises an inlet means, an outlet means, and a microchannel arrangement extending between the inlet and outlet means. The microchannel arrangement can be any microchannel capable of providing a randomized flow path for the biological sample. For example, the microchannel arrangement can include a plurality of transverse separator posts that are integral with a base surface of the microchannel and project therefrom. The posts are generally arranged in a pattern capable of providing a randomized flow path. Examples of microflow apparatuses are described in U.S. application Ser. Nos. 11/458,668 and 11/331,988, both of which are incorporated herein in their entirety. The surface of the microchannel arrangement of the microflow apparatus can be coated partially or entirely, with the ligand.

Exemplary ligands include, but are not limited to, 4′,6′-diamidino-2-phenylindole (DAPI), an acridine, Distamycin, ethidium bromide, 8-methoxypsoralen, diamino-bistetrahydrofuran, an antisense oligonucleotide, a 2′-deoxyribo- or ribonucleotide, a natural or modified oligonucleotide, PNA, LNA, 2′-methoxy-, phosphorothioates, methylphosphonates, or a combination thereof. In one embodiment, the isolated nucleic acid is DNA, and the ligand is a polyclonal anti-DNA antibody, a monoclonal anti-DNA antibody, or a DNA-binding protein.

According to a further aspect of the present invention, it provides a method of identifying the genetic composition of a subject. The method comprises lysing cells in a biological sample of the subject according to a method of the invention, to form a lysing mixture, and identifying the genetic composition of the subject based on a nucleic acid contained in the lysing mixture.

According to yet another aspect of the present invention, it provides a method of identifying the genetic composition of a fetus. The method comprises lysing fetal cells in a maternal biological sample according to a method of the invention, to form a lysing mixture, and identifying the genetic composition of the fetus based on a nucleic acid contained in the lysing mixture.

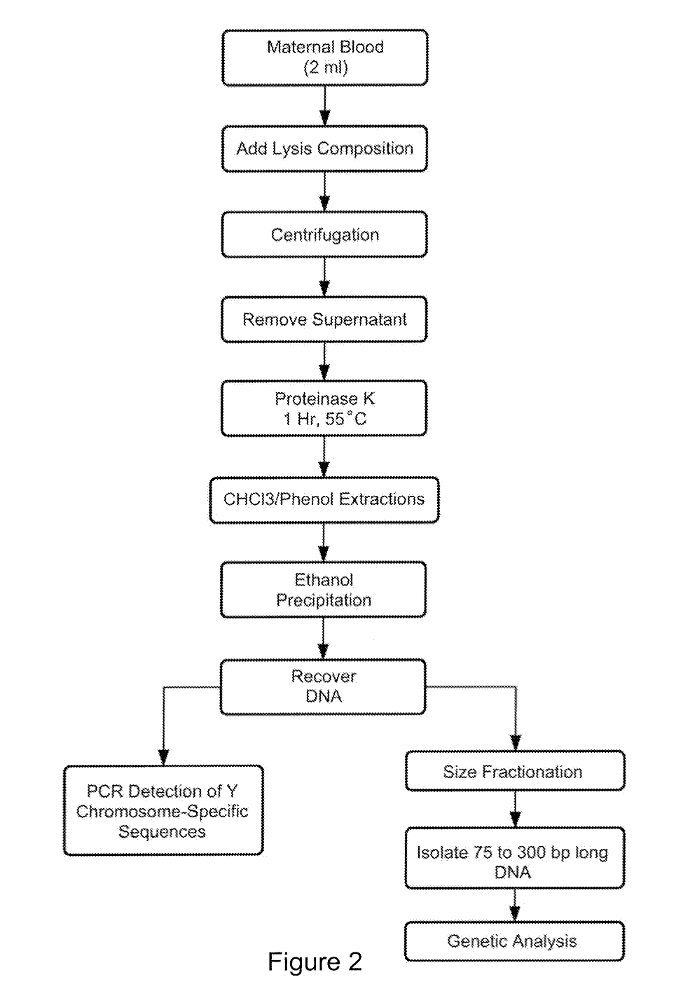

The genetic composition of the fetus can be indicative of the gender of the fetus, or of a condition or disorder in the fetus. In one embodiment, nucleic acids from the lysing mixture can be used directly, i.e., without isolation of fetal nucleic acid from the mixture, to determine the gender of the fetus. In another embodiment, fetal nucleic acid is isolated from the lysing mixture and the genetic composition of the fetus is identified based on the isolated fetal nucleic acid. Fetal nucleic acid can be isolated from the lysing mixture by any known means. In an exemplary embodiment, fetal nucleic acid is isolated from the lysing mixture based on size fractionation. Any known means for size fractionation, e.g., gel electrophoresis (e.g., PAGE), HPLC, TLC, or column-based size fractionation can be used to isolate the fetal nucleic acid. A flow chart depicting exemplary steps of methods of the invention is shown in FIG. 2.

In one embodiment, the genetic composition of the fetus is identified based on the isolated fetal nucleic acid. The genetic composition could be indicative of a condition or disorder in the fetus. Examples of conditions or disorders include, but are not limited to, Cystic Fibrosis, Sickle-Cell Anemia, Beta-thalassemia, Achondroplasia, Preeclampsia, Phenylketonuria, Tay-Scahs Disease, Adrenal Hyperplasia, Fanconi Anemia, Spinal Muscularatrophy, Duchenne's Muscular Dystrophy, Huntington's Disease, Beta Thalassaemia, Myotonic Dystrophy, Fragile-X Syndrome, Down Syndrome, Edwards Syndrome, Patau Syndrome, Klinefelter's Syndrome, Triple X syndrome, XYY syndrome, Trisomy 8, Trisomy 16, Turner Syndrome, Robertsonian translocation, Angelman syndrome, DiGeorge Syndrome, Wolf-Hirschhorn Syndrome, RhD Syndrome, Tuberous Sclerosis, Ataxia Telangieltasia, and Prader-Willi syndrome.

### EXAMPLES

The following examples are intended to illustrate, but not to limit, the invention in any manner, shape, or form, either explicitly or implicitly. While they are typical of those that might be used, other procedures, methodologies, or techniques known to those skilled in the art may alternatively be used.

### Example 1

### Typical Experimental Procedure for Blood Lysis

Maternal blood (2 ml) was treated with 0.2 ml of one of the following compositions for up to 20 minutes at RT.

i. 2.0 mM solution of S-[(2-Guanidino-4-thiazoyl)methyl]-isothiourea hydrochloride in 200 mM HEPES/150 mM NaCl buffer, pH 7.2;
ii. 2.0 mM solution of S-[(2-Guanidino-4-thiazoyl)methyl]-isothiourea hydrochloride in 200 mM HEPES/150 mM NaCl buffer; and 1% Triton X-100;
iii. 2.0 mM solution of S-[(2-Guanidino-4-thiazoyl)methyl]-isothiourea hydrochloride in 200 mM HEPES/150 mM NaCl buffer; and 0.4 mM Vitamin E; or
iv. 2.0 mM solution of S-[(2-Guanidino-4-thiazoyl)methyl]-isothiourea hydrochloride in 200 mM HEPES/150 mM NaCl buffer; 0.4 mM Vitamin E and 1% Triton X-100

The sample was then centrifuged at 1200×g for 10 minutes. The DNA from the supernatant (about 1200 μl) was treated with 300 μl of proteinase K (concentration 10 mg per ml), at 55° C. for one hour. After deactivating the proteinase K by heating the sample at 95° C. for 10 minutes, the sample was extracted with chloroform/phenol (2×500 μl), followed by ethanol precipitation of DNA. The DNA pellet was suspended in 100 μl of nuclease free water and used for PCR and further analysis, e.g., gender determination or genetic composition identification. Approximately 2-20 ng of DNA was used per PCR reaction with two replicates.

### Example 2

### Gender Determination

The DNA from Example 1 was used as a template for determining the gender of the fetus using primers and probes in PCR. Y-chromosome sequences were detected using one or more TaqMan probes, probes that are dual-labeled, 18-22 base oligonucleotide probes with a reporter fluorophore at the 5′-end and a quencher fluorophore at 3′-end, and one or more primers for Y-chromosome sequence markers.

SRY (Sex-determining Region Y) primers were used to target a sex-determining gene on the Y chromosome, present in humans and other primates. The SRY gene encodes the testis determining factor, which is also referred to as the SRY protein. FCY primers were used to target another common marker in the Y chromosome. The beta-hemoglobin gene, a house-keeping gene that is present in total DNA, was used as an internal control in every PCR reaction.

The following controls were used for the PCR reactions:

### Female DNA (Negative Control): 200 ng in 5 μl

### Control Male Genomic DNA (Positive Control):

0 pg control DNA in 5 μl;
7 pg control DNA in 5 μl;
40 pg control DNA in 5 μl
100 pg in μl:
200 pg in μl.

A 96-microwell plate lay-out for 1-11 samples was used for the PCR reaction. Microwells 1-2 and 3-4 contained the primers and probe mix for controls and samples. The reactions for all controls (male DNA as positive control, female DNA as negative control, and beta-globin), and samples were performed in duplicate for each marker:

| Controls with SRY Primers and Probes | | Samples | |
|---|---|---|---|
| 1.5 μM SRY Primer mix: | 2.5 μl | 1.5 μM SRY Primer mix = | 2.5 μl |
| 2.0 μM SRY Probe mix: | 2.0 μl | 2.0 μM SRY Probe mix = | 2.0 μl |
| Male genomic DNA: | 5.0 μl | Extracted Sample DNA = | 8.0 μl |
| Water: | 3.0 μl | Taqman Universal Mix = | 12.5 μl |
| Taqman Universal Mix: | 12.5 μl | | |
| Controls with FCY Primers and Probes | | Samples | |
| 2.0 μM FCY Primer mix: | 2.5 μl | 1.5 μM FCY Primer mix = | 2.5 μl |
| 3.0 μM FCY Probe mix: | 2.5 μl | 2.0 μM FCY Probe mix = | 2.0 μl |
| Male genomic DNA: | 5.0 μl | Extracted Sample DNA = | 8.0 μl |
| Water: | 2.5 μl | Taqman Universal Mix = | 12.5 μl |
| Taqman Universal Mix: | 12.5 μl | | |
| Controls with β-Globin Primers and Probes | | Sample | |
| 3.0 μM β-Globin Primer mix: | 2.5 μl | 3.0 μM β-Globin Primer mix = | 2.5 μl |
| 2.0 μM β-Globin Probe mix: | 2.5 μl | 2.0 μM β-Globin Probe mix = | 2.5 μl |
| Male genomic DNA: | 5.0 μl | Extracted Sample DNA = | 7.5 μl |
| Water: | 2.5 μl | Water = | 2.5 μl |
| Taqman Universal Mix: | 12.5 μl | Taqman Universal Mix = | 12.5 μl |

### PCR Cunning Conditions:

| Step | Temperature | Time | | Cycles |
|---|---|---|---|---|
| Initial Denaturation | 95° C. | 15 | min | 1 |
| Denaturation | 94° C. | 30 | s | 32 |
| Annealing | 57-61° C. | 60 | s | |
| Elongation | 72° C. | 60 | s | |
| Final Elongation | 72° C. | 30 | min | 1 |

The results of gender testing from whole blood from 2 ml of maternal blood from pregnant women (gestation 7 to 12 weeks) is shown in Table 1.

TABLE 1

| | SRY | FCY | Gender by | Concordant |
|---|---|---|---|---|
| Sample # | (Ct Value) | (Ct Value) | RT-PCR | Data |
| 10568 | 34 | 34.5 | Male | Male |
| 10569 | 35.5 | 35.5 | Male | Male |
| 10574 | 34.5 | 35.5 | Male | Male |
| 10575 | SRY negative | FCY negative | Female | Female |
| 10589 | 35 | 33 | Male | Male |
| 10590 | 34.5 | 35.5 | Male | Male |
| 10591 | SRY negative | FCY negative | Female | Female |
| 10592 | 33.5 | 33.5 | Male | Male |
| 10593 | SRY negative | FCY negative | Female | Female |
| 10594 | SRY negative | FCY negative | Female | Female |
| 10595 | 36 | 35 | Male | Male |
| 10615 | 35 | 35 | Male | Male |
| 10617 | 34.5 | 35 | Male | Male |
| 10618 | SRY negative | FCY negative | Female | Female |
| 10619 | 34.5 | 34.5 | Male | Male |
| 10620 | 35 | 34 | Male | Male |

Thus far, we have tested 165 blood samples with PCR using SRY and FCY probes. The gender of 162 samples has been in accordance with the concordant data.

### Example 3

### Use of Size Fractionated Fetal DNA for the Identification of Trisomy-21

The following describes a case study using clinical sample # 11101.

Step 1: Lysis of Blood:

6 ml blood from a pregnant woman (sample # 11101), collected in ACD, arrived at the laboratory within 24 hours of collection. 0.6 ml of one of the lysis compositions of Example 1 was added and the sample was allowed to stand at RT for 20 minutes after thoroughly mixing it with the lysis compositions. After lysis, total DNA was isolated using Roche's MagNAPure kit. The concentration of DNA was determined on a NanoDrop™ (Thermo Scientific). Typically, the yield of DNA was 2 to 4 μg from 6 ml of blood.

Step 2: Size Fractionation on 1.5% Agarose:

Total DNA isolated from maternal blood using the method of Example 1 was fractionated on 1.5% agarose by loading 2 μg DNA per lane. The gel electrophoresis was performed for 90 minutes at 120 volts. The gel was then stained with 0.1% ethidium bromide and visualized under UV light. A typical UV picture of the gel is shown in FIG. 3.

After size fractionation of total DNA, the band marked between the two lines in FIG. 3 (lanes 4-6), corresponding to DNA 50 to 300 bases in length, was excised and extracted with Promega's SV Gel and PCR clean-up kit. The concentration was determined on NanoDrop™ and found to be 2.6 ng per μl.

Step 3: Detection of Fetal Allele and Trisomy-21:

A total of 20 ng (8 μl) of the size fractionated fetal DNA was used as template. PCR was performed in a volume of 25 μl using forward and reverse primers for chromosome-4 and using the PCR components of Table 2. The PCR conditions are described in Table 3.

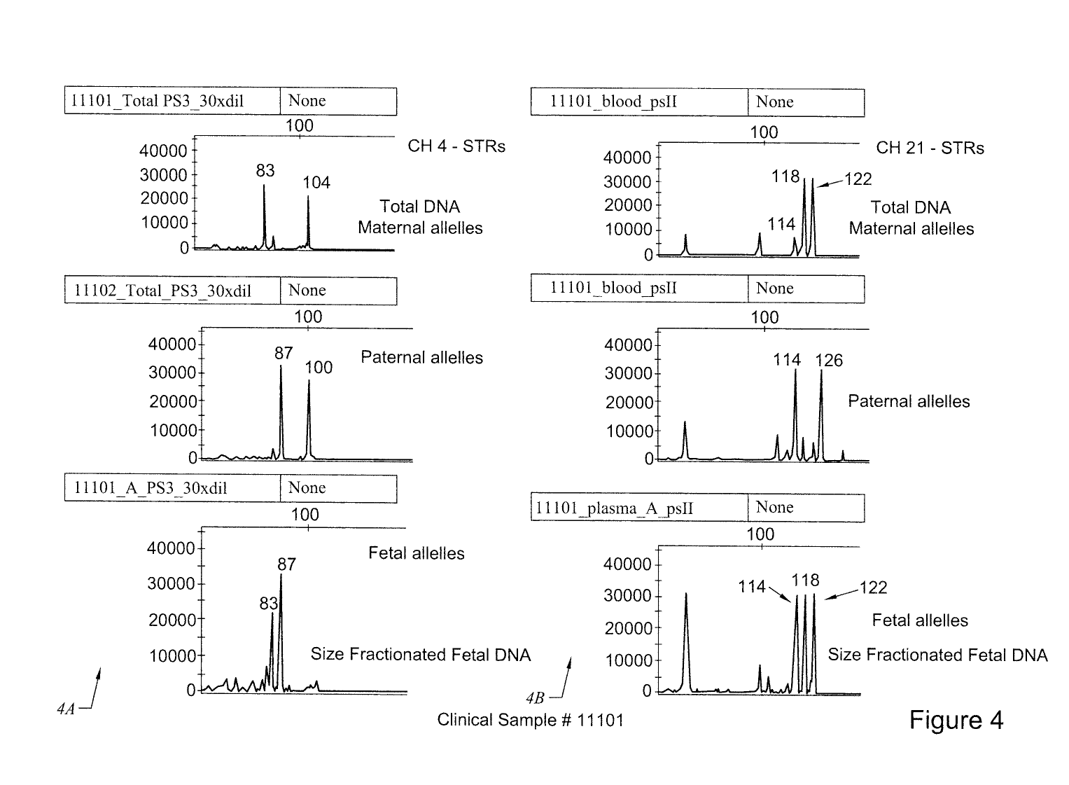

FIG. 4A shows electropherograms of the resulting PCR product with chromosome-4 primers from total maternal DNA, paternal DNA and size fractionated fetal DNA. The fetal allele was determined to be 87 bases long. This allele was common with one of the two paternal alleles that were 87 and 100 bases long. Maternal alleles were 83 and 104 bases long. As shown in the electropherogram of FIG. 4A, the purity of fetal DNA was higher than 95% due to the absence of the second maternal allele (104 bases long) in the size fractionated DNA.

FIG. 4B shows electropherograms of the resulting PCR product using chromosome-21 primers on total maternal DNA, paternal DNA and size fractionated fetal DNA. Maternal alleles were 118 and 122 bases long. Fetal DNA showed three alleles at 114, 118, 122 of equal intensity. This was diagnostic of trisomy-21 DNA (Down syndrome) that had arisen from maternal nondisjunction. The alleles present at 118, and 122, were from the mother and the one present at 114 was from the father.

TABLE 2

| PCR Reaction Components | Volume |
|---|---|
| DNA template | 8.0 μl |
| MgCl2 25 mM | 1.5 μl |
| dNTPs 10 mM | 1.5 μl |
| Forward Primer 50 μM | 0.5 μl |
| Reverse Primer 50 μM | 0.5 μl |
| 10xPCR Gold Buffer | 2.5 μl |
| AmpliTaq Polymerase | 1.0 μl |
| H₂O | 9.5 μl |
| Total | 25 μl |

TABLE 3

| Step | Temperature | Time | | Cycles |
|---|---|---|---|---|
| Initial Denaturation | 95° C. | 15 | min | 1 |
| Denaturation | 94° C. | 30 | s | 36 |
| Annealing | 57-61° C. | 60 | s | |
| Elongation | 72° C. | 60 | s | |
| Final Elongation | 72° C. | 30 | min | 1 |

Although the invention has been described with reference to the presently preferred embodiment, it should be understood that various changes and modifications, as would be obvious to one skilled in the art, can be made without departing from the spirit of the invention. Accordingly, the invention is limited only by the following claims.

1. A composition comprising a solution of S-[(2-Guanidino-4-thiazoyl)methyl]-isothiourea (GTMI), or a salt thereof, at a concentration from about 0.1 mM to about 500 mM, wherein the pH of the solution is from about pH 6 to about pH 9.

2. The composition of claim 1, wherein the concentration of GTMI is from about 0.5 mM to about 100 mM.

3. The composition of claim 1, wherein the concentration of GTMI is from about 0.5 mM to about 25 mM.

4. The composition of claim 1, wherein the concentration of GTMI is from about 1 mM to about 5 mM.

5. The composition of claim 1, wherein the pH of the solution is from about pH 7 to about pH 8.

6. The composition of claim 1, further comprising a buffer at a concentration from about 5 mM to about 500 mM.

7. The composition of claim 1, further comprising a buffer at a concentration from about 10 mM to about 300 mM, and optionally, from about 10 mM to about 300 mM NaCl.

8. The composition of claim 1, further comprising a buffer selected from the group consisting of ([tris(hydroxymethyl)methyl]amino)propanesulfonic acid (TAPS); N,N-bis(2-hydroxyethyl)glycine (Bicine); tris(hydroxymethyl)methylamine (Tris); N-tris(hydroxymethyl)methylglycine (Tricine); N-2-hydroxyethyl-1-piperazineethanesulfonic acid (HEPES); 2-([Tris(hydroxymethyl)methyl]amino)ethanesulfonic acid (TES); (N-morpholino)propanesulfonic acid (MOPS); piperazine-N,N′-bis(2-ethanesulfonic acid) (PIPES); dimethylarsinic acid (Cacodylate); 2-(N-morpholino)ethanesulfonic acid (MES); N-(2-hydroxyethyl)piperazine-N′-2-hydroxypropane-sulfuric acid (HEPPSO); N,N′-Bis(2-hydroxyethyl)-2-aminoethanesulfonicacid (BES); and Phosphate,
wherein the concentration of the buffer is from about 5 mM to about 500 mM.

9. The composition of claim 1, further comprising a buffer, wherein the buffer is HEPES, wherein the concentration of the buffer is from about 10 mM to about 300 mM, and wherein the pH is from about pH 7 to about pH 8.

10. The composition of claim 1, further comprising a buffer, wherein the buffer is HEPES, wherein the concentration of the buffer is about 200 mM, and wherein the pH is about pH 7.2.

11. The composition of claim 4, further comprising a buffer, wherein the buffer is HEPES, wherein the concentration of the buffer is about 200 mM, and wherein the pH is about pH 7.2.

12. The composition of claim 1, further comprising a surfactant.

13. The composition of claim 1, further comprising a non-ionic detergent selected from the group consisting of Triton X-100, Triton X-114, Triton X-405, Dodecyl-beta-D-glucopyrnaside, Dodecyl-beta-D-maltoside, n-Decyl-beta-D-maltopyranside, n-Dodecanoylsucrose, n-Heptyl-beta-D-thioglucopyranoside, n-Hexyl-beta-D-glucopyranside, n-Octanoylsucrose, IGEPAL, Pluronic F-68, HECAMEG, ELUGENT, PLURINIC F-127, Big CHAP, Saponin, Tween-20, Zwittergent 308, 312, 316, and n-Dodecyl-octaethylene glycol (C12E8).

14. The composition of claim 1, further comprising a salt, Triton X-100 or Vitamin E.

15. The composition of claim 10, further comprising about 0.1%-about 10% Triton X-100, about 10 mM-about 300 mM NaCl, or about 0.1 mM-about 2 mM Vitamin E.

16. The composition of claim 11, further comprising about 0.5%-about 5% Triton X-100, about 10 mM-about 300 mM NaCl, or about 0.2 mM-about 1 mM Vitamin E.

17. A method of lysing cells in a biological sample comprising:
contacting a biological sample containing one or more cells with the composition of claim 1.

18. The method of claim 17, wherein the biological sample is blood, plasma, serum, bone marrow, tear, aqueous humour, vitreous humour, saliva, spinal fluid, urine, sputum, mucus, pleural fluid, synovial fluid, sweat, semen, menses, amniotic fluid, cervical mucus or chorionic villus sample.

19. A method of preferentially lysing apoptotic cells in a biological sample comprising:
contacting a biological sample containing apoptotic cells and non-apoptotic cells with a lysing agent for a period of time, wherein the apoptotic cells are preferentially lysed over the non-apoptotic cells.

20. The method of claim 19, wherein the lysing agent is a composition of claim 1.

21. The method of claim 19, wherein the lysing agent is a composition of claim 4.

22. The method of claim 21, wherein the period of time is from about 5 minutes to about 30 minutes.

23. The method of claim 19, wherein the biological sample is blood, plasma, serum, bone marrow, tear, aqueous humour, vitreous humour, saliva, spinal fluid, urine, sputum, mucus, pleural fluid, synovial fluid, sweat, semen, menses, amniotic fluid, cervical mucus or chorionic villus sample.

24. A method of preferentially lysing fetal cells in a maternal biological sample comprising:
contacting a maternal biological sample containing fetal cells with a lysing agent for a period of time, wherein the fetal cells are preferentially lysed over maternal cells in the biological sample.

25. A method of isolating nucleic acids from fetal cells comprising:
contacting a maternal biological sample containing fetal cells with a lysing agent for a period of time to form a lysing mixture and isolating nucleic acid from the lysing mixture, wherein the fetal cells are preferentially lysed over maternal cells in the biological sample.

26. The method of claim 25, wherein the biological sample is a blood, plasma, serum, urine, cervical mucus, amniotic fluid, or chorionic villus sample.

27. The method of claim 25, wherein isolation of nucleic acid from the lysing mixture is carried out using a population of magnetic particles or a surface coated with a ligand for nucleic acids.

28. The method of claim 27, wherein the nucleic acid is DNA, and wherein the ligand is a polyclonal anti-DNA antibody, a monoclonal anti-DNA antibody, or a DNA-binding protein.

29. The method of claim 27, wherein the ligand is 4′,6′-diamidino-2-phenylindole (DAPI), an acridine, Distamycin, ethidium bromide, 8-methoxypsoralen, diamino-bistetrahydrofuran, an antisense oligonucleotide, a 2′-deoxyribo- or ribonucleotide, a natural or modified oligonucleotide, PNA, LNA, 2′-methoxy-, phosphorothioates, methylphosphonates, or a combination thereof.

30. A method of identifying genetic composition of a subject comprising:
lysing cells in a biological sample of a subject according to the method of claim 17 to form a lysing mixture, and identifying genetic composition of the subject based on a nucleic acid contained in the lysing mixture.

31. A method of identifying genetic composition of a fetus comprising:
lysing fetal cells in a maternal biological sample according to the method of claim 24 to form a lysing mixture, and identifying genetic composition of the fetus based on a nucleic acid contained in the lysing mixture.

32. The method of claim 31, further comprising isolating fetal nucleic acid from the lysing mixture.

33. The method of claim 32, wherein fetal nucleic acid is isolated from the lysing mixture based on size fractionation.

34. The method of claim 31, wherein the genetic composition is indicative of the gender of the fetus.

35. The method of claim 31, wherein the genetic composition is indicative of a condition or disorder in the fetus.

36. The method of claim 31, wherein the genetic composition is indicative of a disease or disorder selected from the group consisting of Cystic Fibrosis, Sickle-Cell Anemia, Beta-thalassemia, Achondroplasia, Preeclampsia, Phenylketonuria, Tay-Scahs Disease, Adrenal Hyperplasia, Fanconi Anemia, Spinal Muscularatrophy, Duchenne's Muscular Dystrophy, Huntington's Disease, Beta Thalassaemia, Myotonic Dystrophy, Fragile-X Syndrome, Down Syndrome, Edwards Syndrome, Patau Syndrome, Klinefelter's Syndrome, Triple X syndrome, XYY syndrome, Trisomy 8, Trisomy 16, Turner Syndrome, Robertsonian translocation, Angelman syndrome, DiGeorge Syndrome, Wolf-Hirschhorn Syndrome, RhD Syndrome, Tuberous Sclerosis, Ataxia Telangieltasia, and Prader-Willi syndrome.
